(19) United States
(12) Patent Application Publication
(10) Pub. No.: US 2015/0002812 A1
(43) Pub. Date: Jan. 1, 2015

(54) IMAGE PROCESSING APPARATUS AND STORAGE MEDIUM

(71) Applicant: NIDEK CO., LTD., Gamagori-shi (JP)

(72) Inventors: Yuki YOSHIHARA, Okazaki-shi (JP); Masaaki HANEBUCHI, Nukata-gun (JP); Naohisa SHIBATA, Gamagori-shi (JP)

(21) Appl. No.: 14/316,108

(22) Filed: Jun. 26, 2014

(30) Foreign Application Priority Data
Jun. 27, 2013 (JP) 2013-135626
Jun. 27, 2013 (JP) 2013-135627
Jun. 27, 2013 (JP) 2013-135628

(51) Int. Cl. A61B 3/14; G06T 7/00; A61B 3/00; A61B 3/12

(52) U.S. Cl. CPC A61B 3/14; A61B 3/12; G06T 7/0016; G06T 7/0024; A61B 3/0058; G06T 2200/24; G06T 2207/30041
USPC 351/206

(57) ABSTRACT

An image processing apparatus includes: an analyzer configured to process a plurality of examinee's eye images taken from a same examinee's eye, at least a part of the examinee's eye images being overlapped with each other, and output an analysis result of a cell of the examinee's eye for each of the examinee's eye images; and an instruction receiving unit configured to receive an instruction regarding a target to be analyzed by the analyzer in the plurality of examinee's eye images from an examiner, wherein the analyzer outputs the analysis results in which the instruction received by the instruction receiving unit is reflected for each of the plurality of examinee's eye images.

### CROSS-REFERENCE TO RELATED APPLICATIONS

This application is based upon and claims the benefit of priority from the prior Japanese Patent Applications No. 2013-135626, 2013-135627, and 2013-135628, each filed on Jun. 27, 2013, the entire contents of which are incorporated herein by reference.

### BACKGROUND

This disclosure relates to an image processing apparatus that processes a plurality of images that takes pictures of an examinee's eye, and a storage medium in which program related to the image processing is stored.

### RELATED ART

As an example of an ophthalmic imaging apparatus that takes a picture of an examinee's eye, an apparatus that takes a fundus image by scanning a fundus of the examinee's eye by light and receiving reflected light from the fundus is known (for example, JP-A-2011-115301).

### SUMMARY

Here, one may consider to have an apparatus analyze a state of an examinee's eye by using an image taken by an ophthalmic imaging apparatus. At such an occasion, in order to obtain a more appropriate analysis result, an examiner may want to change an analysis target in the image. Further, for example, in a case of following up the examinee's eye, there may be cases where one wants to have an apparatus analyze a plurality of images that takes pictures of the same examinee's eye. However, in changing the analysis target in the plurality of images, a burden on the examiner tends to become large if the examiner must instruct to change the analysis target for each image.

This disclosure has been made to address the above problems and has a purpose to provide an image processing apparatus that can easily suppress the burden on the examiner who instructs to change the analysis target, and a storage medium in which program related to the image processing is stored.

One aspect of this disclosure provides an image processing apparatus including: an analyzer configured to process a plurality of examinee's eye images taken from a same examinee's eye, at least a part of the examinee's eye images being overlapped with each other, and output an analysis result of a cell of the examinee's eye for each of the examinee's eye images; and an instruction receiving unit configured to receive an instruction regarding a target to be analyzed by the analyzer in the plurality of examinee's eye images from an examiner, wherein the analyzer outputs the analysis results in which the instruction received by the instruction receiving unit is reflected for each of the plurality of examinee's eye images.

A second aspect of this disclosure provides a storage medium storing a computer-readable image processing program, wherein the image processing program, when executed by a processor of a computer, causes the computer to perform: an analyzing step of analyzing plurality of examinee's eye images stored in a storage device, the examinee's eye images having taken a same examinee's eye, and outputting an analysis result of a cell of the examinee's eye for each of the images; and a receiving step of receiving an instruction for changing an analysis condition in the analyzing step from an examiner, in a case where the instruction is received in the receiving step, the analysis results, in which the analysis condition according to the instruction is reflected, are outputted for each of the plurality of examinee's eye images in the analyzing step.

### BRIEF DESCRIPTION OF THE DRAWINGS

FIG. 1 is a block diagram showing a schematic configuration of a PC in an embodiment of the present disclosure;

FIG. 2 is a block diagram showing a schematic configuration of an ophthalmic imaging apparatus of an embodiment;

FIG. 3 is a schematic diagram showing a controller displayed on a monitor;

FIG. 4 is a schematic diagram showing a thumbnail list window;

FIGS. 5A and 5B are schematic diagrams showing wide-field list windows;

FIG. 6 is a flowchart showing an analysis data generating process;

FIG. 7 is a flowchart showing a part of an image adjusting process;

FIG. 8 is a flowchart showing a continued part of the image adjusting process of FIG. 7;

FIGS. 9A to 9C are diagrams for explaining positioning of images in the image adjusting process;

FIG. 10 is a schematic diagram showing a ROI setting window;

FIG. 11 is a flowchart showing a ROI setting process;

FIGS. 12A to 12C are schematic diagrams showing a photoreceptor cell point correcting window;

FIG. 13 is a schematic diagram showing a follow-up window; and

FIG. 14 is an example of a transforming pattern of a display configuration of a photoreceptor cell.

### DETAILED DESCRIPTION OF THE EXEMPLARY EMBODIMENTS

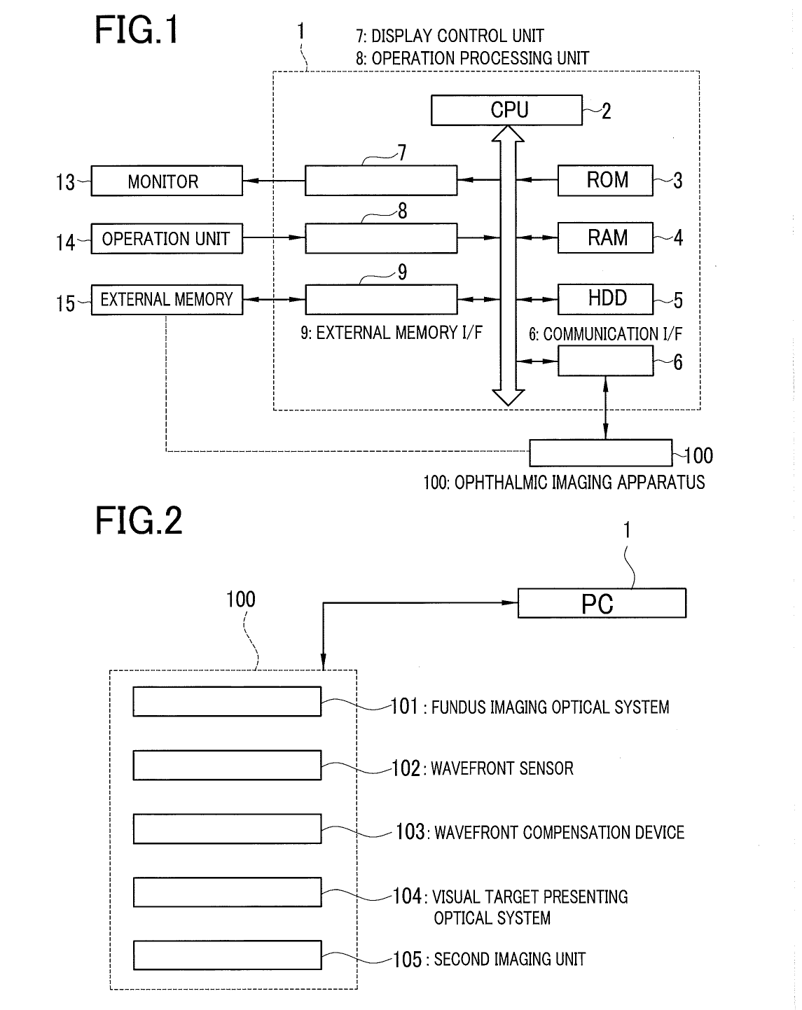

Hereinbelow, an exemplary embodiment of the present disclosure will be described. Firstly, by referring to FIG. 1, a schematic configuration of a personal computer 1 (hereinbelow referred to as “PC 1”) that is an image processing apparatus of the present embodiment will be described.

In the present embodiment, the PC 1 acquires an image of an examinee's eye taken or captured by an ophthalmic imaging apparatus 100 via at least one of a network, an external memory, and the like. The PC 1 performs processing on the acquired image. However, a configuration that can operate as the image processing apparatus is not limited to the PC 1. For example, the ophthalmic imaging apparatus 100 may process the taken image by the ophthalmic imaging apparatus 100 itself. In this case, the ophthalmic imaging apparatus 100 operates as the image processing apparatus.

As shown in FIG. 1, the PC 1 includes a CPU 2. The CPU 2 is a processing device (processor) for executing various processes of the PC 1. The CPU 2 is connected to a ROM 3, a RAM 4, a HDD 5, a communication I/F 6, a display control unit 7, an operation processing unit 8, and an external memory I/F 9 via a bus.

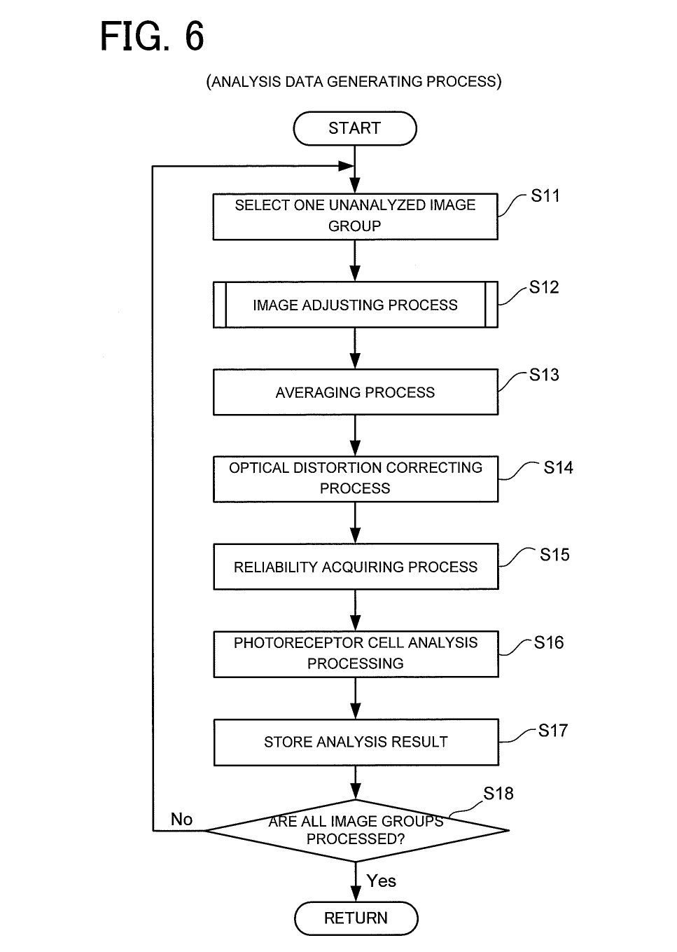

The ROM 3 is a nonvolatile storage medium in which program such as BIOS and the like is stored. The RAM 4 is a volatile storage medium that temporarily stores various types of information. The HDD 5 (Hard Disk Drive 5) is a nonvolatile storage medium. Notably, as a nonvolatile storage medium, other storage medium such as a flash ROM and the like may be used. The HDD 5 stores image processing program for processing the image of the examinee's eye. For example, in the present embodiment, program for causing the PC1 to execute the processes shown in flowcharts of FIG. 6 to FIG. 8 and FIG. 11 is stored in the HDD 5. Further, the HDD 5 stores data of the image taken by the ophthalmic imaging apparatus 100. Notably, in the following description for the sake of convenience, the HDD 5 stores the image data taken from the same examinee's eye.

The communication I/F 6 connects the PC 1 to external apparatuses such as the ophthalmic imaging apparatus 100. The PC 1 of the present embodiment can acquire the data of the image taken by the ophthalmic imaging apparatus 100 via the communication I/F 6. In the present embodiment, the image acquired via the communication I/F 6 is stored in the HDD 5. The external memory I/F 9 connects an external memory 15 to the PC 1. As the external memory 15, various types of storage medium such as a USB memory and a CD-ROM may be used.

The PC 1 of the present embodiment can acquire the data of the image taken by the ophthalmic imaging apparatus 100 via the external memory 15. For example, a user can attach the external memory 15 to the ophthalmic imaging apparatus 100, and store the data of the image taken by the ophthalmic imaging apparatus 100 in the external memory 15. Then, the user attaches the external memory 15 to the PC 1, and causes the PC 1 read the image data stored in the external memory 15. As a result, the PC 1 acquires the data of the image taken by the ophthalmic imaging apparatus 100.

Here, by referring to FIG. 2, a schematic configuration of the ophthalmic imaging apparatus 100 of the present embodiment will be described. In the present embodiment, a case of using a wavefront compensated laser ophthalmoscopy device (AO-SLO) as the ophthalmic imaging apparatus 100 will be described. The ophthalmic imaging apparatus 100 includes a fundus imaging optical system 101, a wavefront sensor 102, a wavefront compensation device 103, an visual target presenting optical system 104, and a second imaging unit 105. Notably, detailed configurations of the ophthalmic imaging apparatus 100 can be exemplified in the contents described in JP-A-2013-070941.

The fundus imaging optical system 101 two dimensionally scans illumination luminous flux (laser light) on a fundus of the examinee's eye. Further, the fundus imaging optical system 101 receives reflected light (reflected luminous flux) reflected at the fundus and acquires an image of the examinee's eye (that is, a fundus image). According to this, the fundus imaging optical system 101 images the fundus with high resolution (high discrimination) and high magnification. In the present embodiment, in order to make observation and the like at a cellular level, the image is taken at an image angle of about 1.5 degrees. The fundus imaging optical system 101 can change an imaging portion by moving an illumination luminous flux scan area of the examinee's eye in up, down, left, and right directions. Further, in the present embodiment, the fundus imaging optical system 101 takes images sequentially of the same range. In the present embodiment, for example, the fundus imaging optical system 101 can take about 150 images by sequentially taking images for about 3 seconds. That is, in the fundus imaging optical system 101, a series of picture taking can acquire one group of images including a plurality of sequential still images. Notably, the fundus imaging optical system 101 for example can be configured of a can-type laser ophthalmoscope using a confocal optical system.

The data of the image taken by the fundus imaging optical system 101 is acquired by the PC 1 according to the above described method. The image data to be acquired by the PC 1 includes gradation information, coordinate information, and the like as data for forming an image. Other than the aforementioned, in the present embodiment, the image data for example includes an ID of the examinee's eye, a time stamp indicating a date on which the image is taken, information indicating the taken portion within the fundus, information indicating the presented position of the fixation target upon taking the image, and the like.

In the ophthalmic imaging apparatus 100 of the present embodiment, in a case where the fundus image is to be taken, a wavefront aberration by the examinee's eye is compensated using the wavefront sensor 102 and the wavefront compensation device 103. The wavefront sensor 102 is an element for detecting the wavefront aberration including a low order aberration and a high order aberration. In the present embodiment, the wavefront sensor 102 receives the reflected luminous flux reflected by the fundus and detects the wavefront aberration of the examinee's eye. As the wavefront sensor 102, for example, a Hartmann-shack detector, a wavefront curvature sensor that detects a change in light intensity and the like can be used.

The wavefront compensation device 103 relays the illumination light irradiated to the examinee's eye by the fundus imaging optical system 101. At such an occasion, the wavefront compensation device 103 deforms a reflecting surface of the illumination light based on a detection result of the wavefront sensor 102 for example. Due to this, the wavefront compensation device 103 controls the wavefront of the illumination light to compensate the wavefront aberration by the examinee's eye. As the wavefront compensation device 103, for example, a reflection type LCOS (Liquid Crystal On Silicon), a deformable mirror and the like can be used.

The visual target presenting optical system 104 presents a fixation target to the examinee's eye upon taking the image of the fundus by the ophthalmic imaging apparatus 100. In the ophthalmic imaging apparatus of the present embodiment, the visual target presenting optical system 104 can switch the presented position of the fixation target. In the present embodiment, the presented position of the visual target is set at a total of 9 portions, namely in three rows each in the up and down direction and the left and right direction of the examinee's eye. The area in which the illumination light can be irradiated in the fundus is changed by switching the presented position of the visual target and guiding the sight of the examinee's eye. Notably, the sight of the examinee's eye can be guided by moving the fixation target by the visual target presenting optical system 104.

The second imaging unit 105 acquires a fundus image with a wider angle than the fundus imaging optical system 101 (that is, a wide field image). The fundus image acquired by the second imaging unit 105 for example is used as an image for designating or confirming the position of the fundus taken by the fundus imaging optical system 101. The second imaging unit 105 can use a known observation and imaging optical system of a fundus camera, or optical system of a scanning laser ophthalmoscope (SLO). Although the details will be described later, the apparatus of the present embodiment causes the PC 1 to acquire not only the images taken by the fundus imaging optical system 101, but also the fundus images taken by the second imaging unit 105. At this occasion, in the PC 1, the wide angle fundus image taken while having the same presented position of the fixation target as the group of images acquired by the PC 1 is at least acquired. Due to this, the HDD 5, the external memory 15, and the like of the PC 1 can store the wide angle fundus image.

Returning to FIG. 1, the description of the image processing apparatus 1 will be continued. The display control unit 7 controls display on a monitor 13. The operation processing unit 8 is connected to an operation unit 14 (for example, a keyboard, a mouse, and the like). The operation processing unit 8 detects an operation on the operation unit 14 by a user and outputs an operation signal to the CPU 2. Due to this, the user's operation on the operation unit 14 is received by the CPU 2. Notably, in the present embodiment, the externally connected monitor 13 and operation unit 14 are used. However, at least a part of the monitor 13 and operation unit 14 may be installed in the PC 1.

In the present embodiment, the user's operation on the operation unit 14 is performed on various types of GUI displayed on the monitor 13. As one type of the GUI, a controller 20 for mainly receiving the user's operation is displayed on the monitor 13.

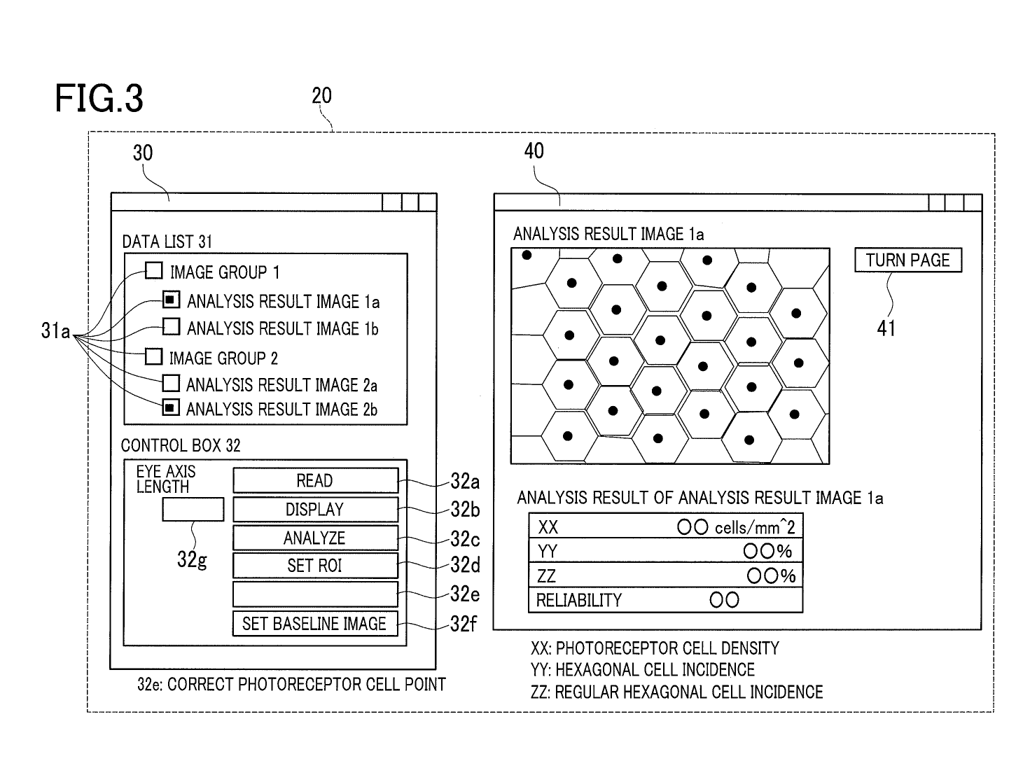

Here, a schematic configuration of the controller 20 will be described with reference to FIG. 3. A setting window 30 is displayed in the controller 20. The setting window 30 includes a data list 31 and a control box 32. The data list 31 displays a list of the images stored in the HDD 5 and external memory 15. The data list 31 displays a list of file names of each group of images (for example, “Image group 1”, “Image group 2”). In the present embodiment, the image taken by the fundus imaging optical system 101 that the PC 1 acquired from the ophthalmic imaging apparatus 100 is managed by being given one file name for each group of images. As mentioned above, each group of images includes the plurality of still images that are taken sequentially by the fundus imaging optical system 101.

Further, as shown in FIG. 3, in the data list 31, a list of file names of analysis result images generated from the group of images is displayed under the file name of the group of images. For example, in FIG. 3, “Analysis result image 1a” and “Analysis result image 1b” generated from the “Image group 1” are displayed under the “Image group 1”. Although the details will be described later, the analysis result images are still images used for a photoreceptor cell analysis of the examinee's eye. Accordingly, in the present embodiment, the lists of the groups of images and the file names of the analysis result images are displayed in the data list 31.

A check box 31a is provided at a header portion of each of the file names displayed in the data list 31. In the apparatus of the present embodiment, a check operation by the user (for example, clicking by the mouse) performed on each check box 31a is received. The user can select the image on which a processing such as display is to be performed by checking the check box 31a.

The control box 32 includes a plurality of buttons and input columns. Although the details will be described later, a window may be expanded in the controller 20, or a parameter to be used for the photoreceptor cell analysis is changed in accordance with an operation by the user on the buttons and input columns. For example, when a “DISPLAY” button 32b is operated in a state where one of the images in the data list 31 is selected, an image display window 40 as shown in FIG. 3 is expanded in the controller 20.

The image selected in the data list 31 is displayed in the image display window 40. In the present embodiment, as shown in FIG. 3, in a case where a plurality of images is selected in the data list 31, one of the images is displayed in the image display window 40. At this occasion, the image displayed in the image display window 40 can be switched by operating a “TURN PAGE” button 41. Notably, in a case where the analysis result image is selected in the data list 31, a result of the analysis processing performed by using the analysis result image is displayed in the window 40. Further, in a case where a group of images is selected in the data list 31, each still image included in the group of images is displayed sequentially in a taken order. That is, a movie is displayed in the image display window 40. Notably, the monitor 13 may display a plurality of selected images in a case where the “DISPLAY” button 32b is operated in the state where the plurality of image is selected in the data list 31.

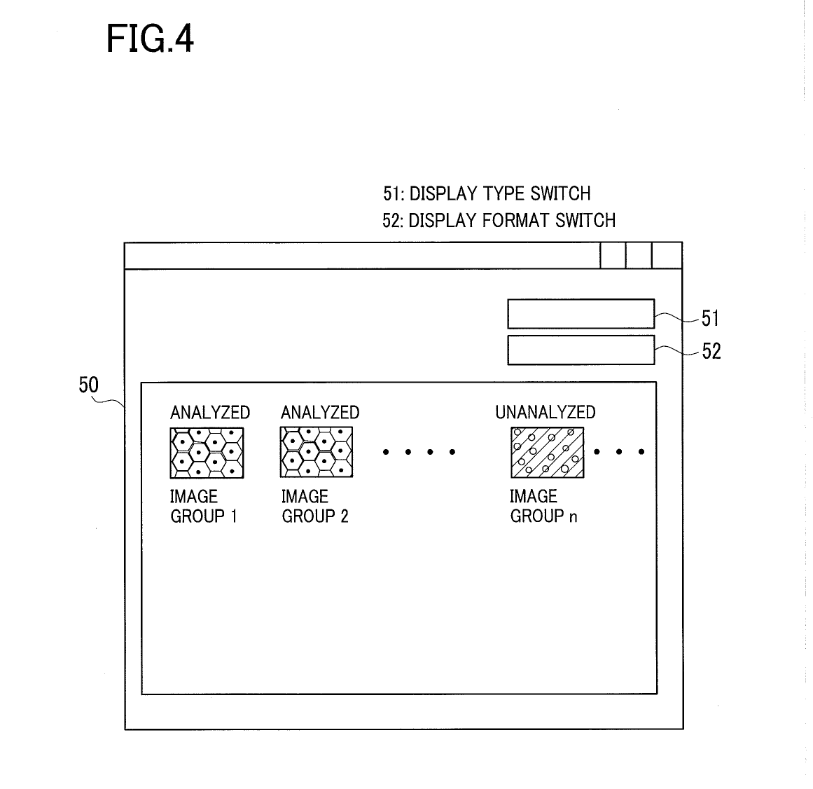

Next, an operation of the PC 1 will be described by referring to FIG. 4.

<Image Selection>

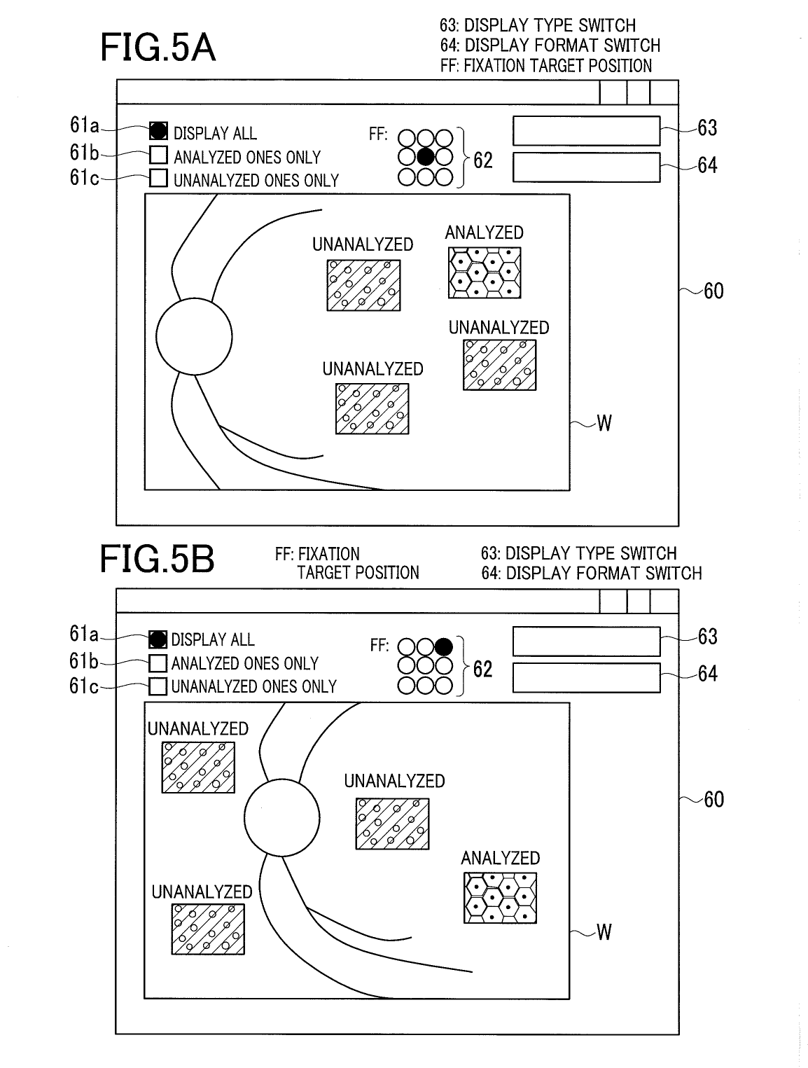

As mentioned above, the CPU 2 selects an image on which an image processing such as analysis, or a processing to display in the monitor 13 is to be performed based on the user's operation on the check box 31a of the data list 31. The PC 1 of the present embodiment has other methods for selecting an image to be used in the processing such as the analysis prepared therein. For example, an image to be used in the processing such as the analysis can be selected from a thumbnail list window 50 shown in FIG. 4 and wide-field list windows 60 shown in FIGS. 5A and 5B.

In the thumbnail list window 50 shown in FIG. 4, a list of thumbnail images (an example of image index) of the groups of images of which the file names are displayed in the data list 31 is displayed. In a case where an operation on a “READ” button 32a on the control box 32 is received by the apparatus, the thumbnail list window 50 is displayed by the CPU 2. In a case where a “DISPLAY TYPE SWITCH” button 51 is operated by the user, a type of the thumbnail images displayed on the screen is switched by the CPU 2. For example, when the “DISPLAY TYPE SWITCH” button 51 is operated during when the thumbnail images of the groups of images as shown in FIG. 4 are displayed, the thumbnail images displayed in the thumbnail list window 50 switch to the thumbnail image of the analysis result image. Notably, in the present embodiment, a reduced image of one of the still images included in the group of images is used as the thumbnail image of indicating the group of images to which the analysis processing of the photoreceptor cell is not being performed. For example, the reduced image of the top image taken first among each group of images can be used as the thumbnail image. Further, as the thumbnail image of the group of images to which the analysis processing of the photoreceptor cell has already been performed, a reduced image of the analysis result image generated in the course of the analysis is used. The thumbnail images may be stored in the HDD 5 and the like in advance. Further, the PC 1 may create the thumbnail image in the case where the “READ” button 32a is operated.

As shown in FIG. 4, the CPU 2 displays information indicating a history of the analysis processing of the photoreceptor cell (for example, presence or absence of the analysis processing) together with the thumbnail image in the thumbnail list window 50. In FIG. 4, a display of “Analyzed” indicates that the analysis of the group of images corresponding to the thumbnail image with this indication has already been performed, and the analysis result thereof is stored in the HDD 5 and the like. Further, a display of “Unanalyzed” indicates that the analysis of the group of images corresponding to the thumbnail image with this indication has not yet been performed. Due to this, when the thumbnail list window 50 is displayed, the user can easily understand whether or not the analysis processing has been performed on the groups of images indicated by the thumbnail images. Notably, in the present embodiment, the display of “Analyzed” and “Unanalyzed” was mandatorily displayed together with each of the thumbnail images; however, only one of the display of “Analyzed” and “Unanalyzed” may be displayed.

The wide-field list windows 60 shown in FIGS. 5A and 5B are displayed by being switched from the thumbnail list window 50 when a “DISPLAY FORMAT SWITCH” button 52 is operated in the thumbnail list window 50. The CPU 2 displays the wide angle fundus image W (wide field fundus image W) in an entire thumbnail display area in each wide-field list window 60. The wide field fundus image W is a fundus image taken by the second imaging unit 105 of the ophthalmic imaging apparatus 100. As described above, in the present embodiment, the wide field fundus image W is an image that the PC 1 acquired in advance from the ophthalmic imaging apparatus 100.

The CPU 2 displays the thumbnail image by overlaying the same over the wide field fundus image W. As this occasion, the CPU 2 determines positions to arrange the thumbnail images in accordance with a positional relationship of imaging portions of the images (groups of images, or analysis result image) displayed by the thumbnail images. For example, in FIGS. 5A and 5B, the CPU 2 causes positions where each of the thumbnail images of the groups of images are arranged in the wide field fundus image W to coincide with imaging portions (taken areas) of each group of images. The positioning of the thumbnail images relative to the wide angle fundus image can for example be performed based on information indicating the taken portions within the fundus included in the respective image data. Notably, as the information indicating the taken portions within the fundus, for example, position information relative to an image center of the wide angle fundus image, position information relative to macula, and the like can be used.

In the present embodiment, in a case where there is a plurality of group of images taken at the same portion, the thumbnail images of the group of images are displayed at the same position in the wide field fundus image W in an overlaid manner. If a plurality of thumbnail images is overlapped, file name and the like (an example of an image index) of the group of images indicated by each thumbnail image is displayed around the thumbnail images. By the user's selection operation being performed on the file names on the screen (for example, clicking by the mouse), the CPU 2 can select independent groups of images even in the state where the plurality of thumbnail images is overlapped.

As shown in FIGS. 5A and 5B, check boxes 61a to 61c for selectively displaying the thumbnail images according to an analysis circumstance is provided in the wide-field list window 60. In FIGS. 5A and 5B, the check box 61a of “Display all” is selected. When the check box 61b of “Analyzed ones only” is selected, the CPU 2 displays the thumbnail images indicating the groups of images of which analysis has been completed. In this case, the CPU 2 does not display the thumbnail images indicating the groups of images of which analysis has not yet been performed. Further, when the check box 61c of “Unanalyzed ones only” is selected, the CPU 2 displays the thumbnail images indicating the groups of images of which analysis has not yet been performed. In this case, the CPU 2 does not display the thumbnail images indicating the groups of images of which analysis has been completed. Due to this, in the PC 1, the user can easily select one of the unanalyzed group of images and the analyzed group of images.

Further, in the wide-field list window 60, a list display of the thumbnail images is conducted for each of the presented positions of the fixation targets that were presented upon taking images of the group of images. The wide-field list window 60 has a fixation target position selecting/displaying box 62 provided therein. In the present embodiment, the fixation target position selecting/displaying box 62 has a total of 9 boxes of check boxes, namely three boxes each in the up and down direction and the left and right direction. The 9 boxes of check boxes respectively correspond to the presented positions of the fixation target in the visual target presenting optical system 104 of the ophthalmic imaging apparatus 100. The user can check (select) one of the check boxes to instruct the thumbnail image to be displayed on the screen. When one of the check boxes is checked (selected), the CPU 2 displays the thumbnail images of a group of images taken at the fixation position corresponding to the checked position on the screen. For example, as shown in FIG. 5A, if the check box at the center of the 9 boxes is checked, the thumbnail images of the group of images taken when the presented position of the fixation target was at the center are displayed. Further, as shown in FIG. 5B, if the check box at the upper right side of the 9 boxes is checked, the thumbnail images of the group of images taken with the fixation target presented at the right upper presented position are displayed.

Further, as shown in FIGS. 5A and 5B, in the present embodiment, the wide field fundus image W displayed in the wide-field list window 60 is also selected cooperatively with the checking of the check box. The CPU 2 displays the wide field fundus image W taken at the fixation position corresponding to the checked position in the wide-field list window 60 from a plurality of fundus images taken in advance at the respective fixation positions.

A “DISPLAY TYPE SWITCH” button 63 has the same role as the “DISPLAY TYPE SWITCH” button 51 of the thumbnail list window 50. Further, in a case where a “DISPLAY FORMAT SWITCH” button 64 in the wide-field list window 60 is operated by the user, the display is switched to the thumbnail list window 50 by the CPU 2.

According to the above, in the wide-field list window 60 of the present embodiment, the thumbnail images (one example of the image index) indicating the groups of images or the analysis result image are arranged at the positions corresponding to the taken positions of the respective images on the wide field fundus image W. Due to this, the user can easily understand which positions of the examinee's eye are taken in the groups of images and the like indicated by the thumbnail images. Further, due to this, the user can easily select the groups of images and the like to be used in the image processing.

Further, the group of images and the like having different presented positions of the fixation target from one another despite the taken position in the examinee's eye being the same may in some cases desirably be dealt separately. For example, in the AO-SLO, even for two or more images taken at the same portion, if the position of the fixation target at the time of taking the image is different for each image, there is a risk that a content of each image might be different. With respect to this, in the wide-field list window 60, the thumbnail image is displayed by being switched for each of the fixation target position at the time of taking the image of the group of images and the like indicated by each of the thumbnail images. Accordingly, in the PC 1, the user can easily select the desired group of images even if the groups of images and the like taken at different fixation positions are stored in the HDD 5 and the like.

Notably, the groups of images and the like having different presented positions of the fixation target upon taking the images may be displayed by being overlapped on one wide field fundus image W. In this case, for example, the position of each image in the wide field fundus image W is determined based on the information indicating the presented position of the fixation target as included in the image data, and the information indicating the taken position.

Further, in the present embodiment, each time the display of the thumbnail image is switched for each fixation position, the wide field fundus image W displayed in the wide-field list window 60 switches to the image taken at the same fixation position as the group of images and the like indicated by the thumbnail image. Thus, the user can more appropriately understand the taken position of the group of images and the like indicated by the thumbnail image.

Further, in the wide-field list window 60, similar to the thumbnail list window 50, information indicating the history of the analysis processing of the photoreceptor cell is displayed together with the thumbnail image by the CPU 2. Thus, in the PC 1, the user can easily understand whether or not the analysis processing has been performed on the group of images indicated by the thumbnail image.

Notably, in the present embodiment, the thumbnail image and the file name were exemplified as the image index indicating an image (group of images or analysis result image), however, an icon, taken date, image quality, reliability, and the like, or other information specifying the image may be used as the image index.

<Image Analysis>

If an “ANALYZE” button 32c (see FIG. 3) is operated by the user in the case where one or more groups of images are selected by the aforementioned method, an analysis data generating process (see FIG. 6) is performed by the CPU 2. In the analysis data generating process, an averaging image is generated from each group of images. Further, the analysis processing is performed on each averaging image, and an analysis result is derived.

Here, the analysis data generating process will be described by referring to FIG. 6. Firstly, the CPU 2 selects a group of images to which the processing has not yet been performed by the analysis data generating process as a current processing target among the groups of images selected by the data list and the like (S11). Here, the group of images selected in the process of S11 is indicated by a dataset L (image set L)=[L0, L1, . . . , LN]. Each image is indicated by Ln. However, the Ln with smaller value of the subscript n indicates that the image is taken at an earlier time.

Next, the CPU 2 performs an image adjusting process (S12). In the present embodiment, in the image adjusting process, a base image (first base image) is generated from a part of or all of images taken by the ophthalmic imaging apparatus 100 when fixation is stabilized, among the plurality of images included in the group of images selected by the process of S11. In the image adjusting process (S12) of the present embodiment, the base image is used as a template for correcting a difference between images by overlapping a part of all of the images of the group of images. Although the details will be described later, the image having the difference between images being adjusted by the image adjusting process is subjected to averaging in the process of subsequent S13.

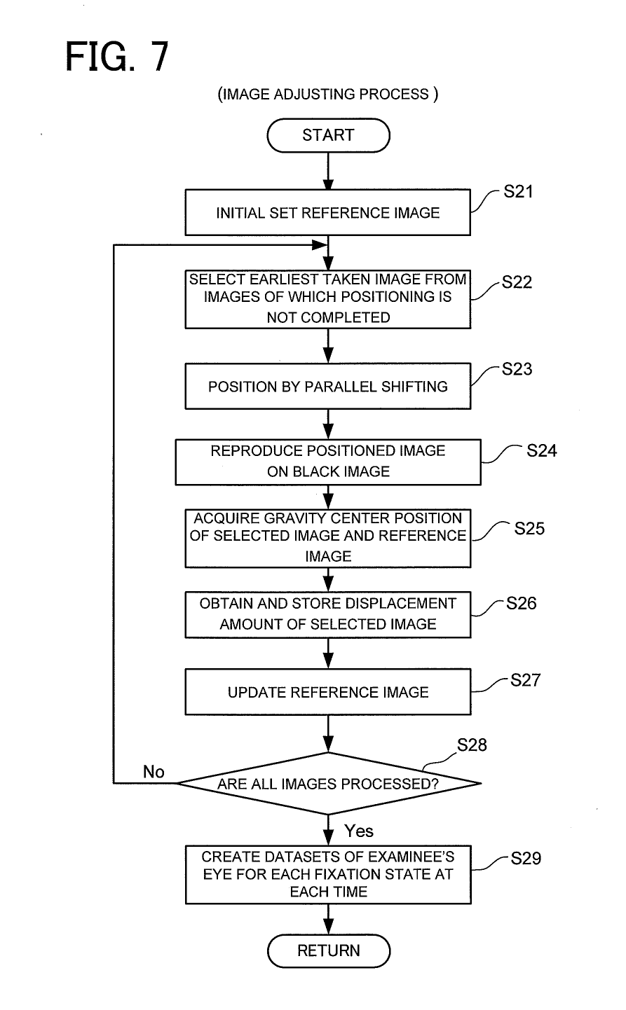

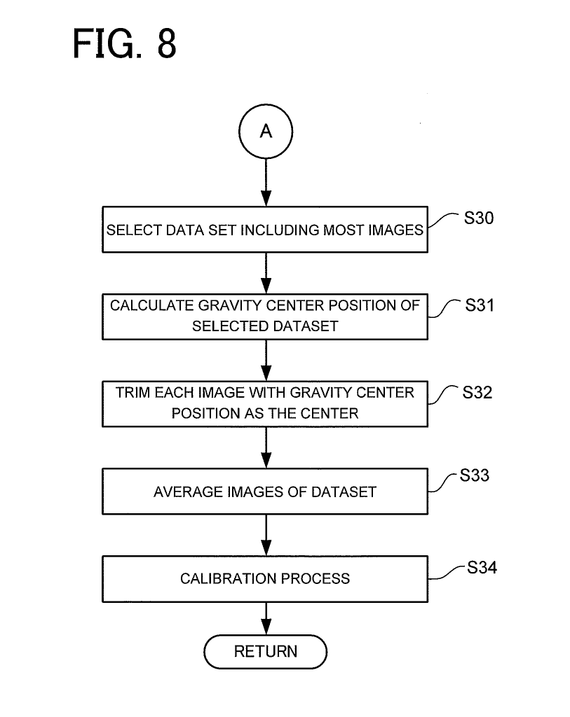

Here, the image adjusting process will be described by referring to FIG. 7 and FIG. 8.

Firstly, the CPU 2 performs the processes from S21 to S28 to perform a rough positioning of the images included in the group of images (that is, the dataset L) selected in the process of S11. The rough positioning referred herein means positioning performed at least without correcting distortions in each image. In the present embodiment, the rough positioning is performed by shifting the fundus image Ln in parallel. However, the rough positioning is not limited to the parallel shifting, but for example may be a rotative shifting, or a combination of the parallel shifting and the rotative shifting. The rough positioning is performed on a black image E. The size of the black image E is defined by a lateral width Mw and a vertical width Mh. In the above, w is a lateral width of the fundus image Ln, and h is a vertical width of the fundus image Ln. M is a constant of 1 or more (for example, M=3), and defines a range that allows positional displacement between images. Further, the CPU 2 stores the images after the rough positioning and displacement amounts of the images (details of which will be described later) in the RAM 4. The respective image dataset to which the rough positioning has been performed will be indicated by G=[G0, G1, . . . , GN].

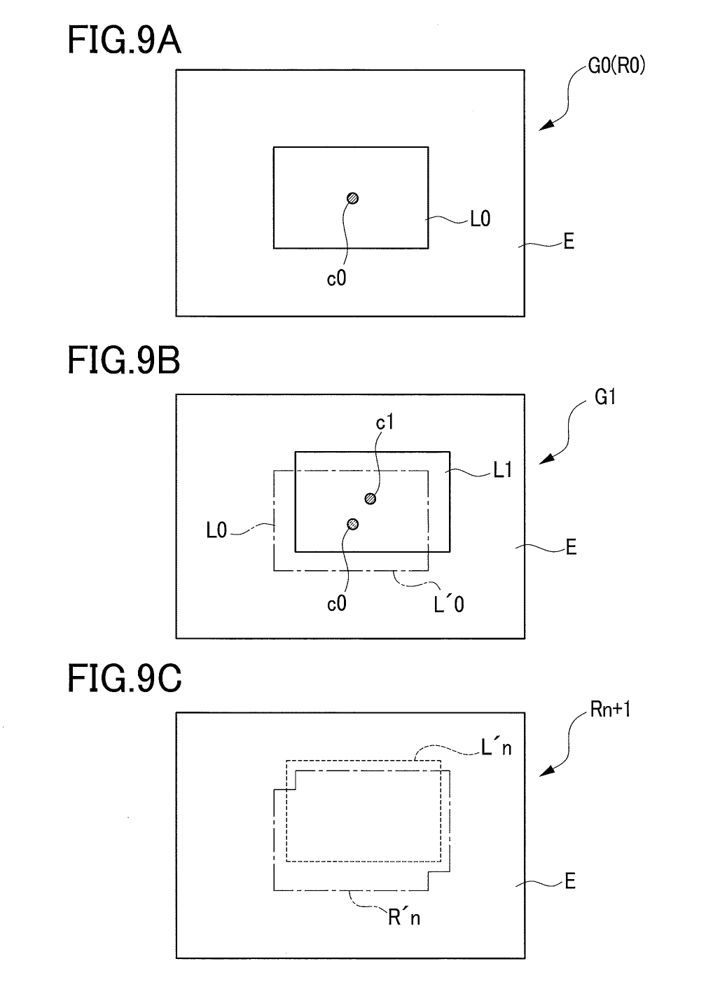

In the process of S21, an initial setting of a reference image R (second base image) is performed by the CPU 2 (S21). The reference image R is used as a reference for roughly positioning the fundus images Ln. Although the details will be described later, in the present embodiment, the reference image R is updated each time the fundus images Ln are positioned relative to the reference image R. Hereinbelow, the reference image after n times of updating will be indicated by Rn. The reference image R0 to be initially set in the present embodiment is, as shown in FIG. 9A, an image in which an image L0 is arranged on the black image E such that its center of gravity position overlaps with that of the black image E.

After S21 is performed, the CPU 2 repeatedly performs the processes of S22 to S28, and roughly positions the images included in the dataset L relative to the reference Image Rn on one-by-one basis. Firstly, the image Ln of which rough positioning is not completed and having the earliest image-taking time is selected by the CPU 2 as the image to be positioned next (S22). For example, in a case where an image Lk was positioned in the processes of S22 to S28 that had just been performed, an image Lk+1 is selected by the CPU 2 in the subsequent process of S22. Notably, in the process of S22 just after the process of S21 having been performed, the image L0 is selected by the CPU 2.

In the subsequent process of S23, the image Ln (hereafter referred to as “Selected image”) selected in the previous process of S22 is positioned to the reference image Rn by the parallel shifting (S23). Various types of image processing methods can be used as the method of positioning. For example, a method by which the selected image Ln may be subjected to positional displacement on one pixel at a time relative to the reference image Rn, and the selected image Ln is positioned at a position with the highest match between both images (position with the highest correlation) may be considered. Further, a method by which mutual characteristic points are extracted from the reference image Rn and the selected image Ln, and the selected image Ln is positioned at a position where the characteristic points of each other overlap may be considered.

In the present embodiment, the positioning is performed by successively calculating a correlation value of the selected image Ln and the reference image Rn while displacing the selected image Ln in one pixel units relative to the reference image Rn. Notably, the maximum value of the correlation value is 1, and a larger value indicates that the correlation between the images is higher. Next, the CPU 2 generates an image Gn (S24) by reproducing the selected image Ln, which was moved to the position where the correlation with Rn becomes the highest, on the black image E. For example, in a case of positioning the image L0, the image L0 completely matches the fundus image portion included in the reference image R0 upon the initial setting. Thus, an image G0 comes to be the same as the reference image R0 (see FIG. 9A). Further, as shown in FIG. 9B, in an image G1, L1 is moved such that overlapping ranges of a fundus image portion L′0 in the image G0 and the image L1 are overlapped, and then the image L1 is reproduced on the black image E. The generated images Gn is stored in the RAM 4 by the CPU 2.

Further, at this occasion, the CPU 2 acquires a gravity center position cn of the selected image Ln moved to the position with the highest correlation with Rn (S24). Moreover, the CPU 2 stores a positional displacement amount (shifted amount) dn=[dxn, dyn] of the selected image Ln in the RAM 4 (S25). In the present embodiment, the positional displacement amount dn indicates a displacement of the taken areas of the selected image Ln and the selected image Ln−1 taken just before Ln. The displacement of the taken areas is caused by an involuntary eye movement during fixation, so the positional displacement amount dn indicates size and direction of the involuntary eye movement during fixation generated since when the selected image Ln−1 is taken until when the selected image Ln is taken. Thus, the CPU 2 can detect the movement of the examinee's eye upon taking the images based on the displacement amount dn. Notably, in the present embodiment, the positional displacement amount dn of the selected image Ln is set as the displacement of the selected image Ln and the taken image Ln−1 taken at a time earlier than Ln, however, it may be set as a displacement from an image taken at a time after Ln. dxn and dyn respectively indicate a horizontal direction component and a vertical direction component in the positional displacement amount. In the present embodiment, the positional displacement amount do can be obtained for example from a difference between the gravity center position cn and a gravity center position cn−1 that was predeterminedly acquired.

Next, the CPU 2 updates the reference image Rn (S27). In the present embodiment, the CPU 2 generates an updated reference image Rn+1 from the reference image Rn and the roughly-positioned selected image Gn. For example, the reference image Rn and the averaging image of the image Gn can be formed to be the updated reference image Rn+1. In this case, a gradation value rn+1 of a pixel at a voluntary position in the updated reference image Rn+1 can be expressed for example by the following formula (I).

rn+1={(n×rn)+gn}/(n+1) (1)

Notably, rn, r0, and gn respectively indicate the gradation values of the pixel at the same position as the above-mentioned voluntary position in the reference image Rn, the reference image R0 upon the initial setting, and the image Gn. Due to this, as shown in FIG. 9C, the updated reference image Rn+1 has the fundus image portion R′n of the reference image Rn and the fundus image portion L′n of the image Gn averaged.

Next, the CPU 2 determines whether the positioning of all of the images included in the dataset L has been completed or not (S28). If an image of which positioning has not yet been performed still exists in the dataset L (S28: No), the CPU 2 returns to the process of S22 and repeatedly performs the processes from S22 to S28. On the other hand, if the positioning of all of the images included in the dataset L has been completed (S28: Yes), the CPU 2 proceeds to the process of S29. Notably, in the present embodiment, in the processes from S22 to S28, the positioning with the reference image among the images included in the dataset L was performed from images with earlier image-taken time, however, the positioning with the reference image may be performed from images with later image-taken time.

In the process of S29, the CPU 2 divides the dataset G=[G0, G1, . . . , Gn] of the group of images to which the rough positioning has been performed, and creates a plurality of datasets: dataset F1=[G0, G1, . . . , Ga], F2=[Ga+1, . . . , Gb], . . . , Fq=[ . . . , GN] that corresponds chronologically to fixation states of the examinee's eye. Here, a dividing method of the dataset G of the present embodiment will be described. In the present embodiment, the dataset is divided by using the displacement amount dn obtained in the process of S26. For example, in the present embodiment, the displacement amount dn corresponding to the image Gn included in the dataset G is integrated in an order of subscripts (that is, in an image-taken order of the image Ln). Notably, as described earlier, the displacement amount dn expresses a magnitude of the positional displacement in an image-taken range by the involuntary eye movement during fixation caused during when two sequential images are taken by the ophthalmic imaging apparatus 100. Thus, an integrated value S indicates a magnitude of the positional displacement in an image-taken range by the involuntary eye movement during fixation from a certain time. A dataset Fm formed of images Gn having the displacement amounts gn included in the integrated value S is divided from the dataset G at an occasion when the integrated value S of the displacement amounts gn exceeded a predetermined threshold Θ. A plurality of datasets F1, F2, . . . , Fq is created from the dataset G by a similar process being performed on the rest of the dataset G with the integrated value of the displacement amount being initialized (set to zero). Here, the number of images Gn included in the dataset Fm is assumed to be indicating a degree of stability of the fixation of the examinee's eye upon taking the images Gn included in the dataset Fm. This is because the number of images that can be taken by the ophthalmic imaging apparatus 100 during when the image-taken range is positionally displaced by Θ is expected to be increased in cases with greater stability of the fixation, that is, in cases with small chronological change in the image-taken range. Thus, the datasets F1, F2, . . . , Fq created in the process of S29 respectively correspond chronologically to the fixation states of the examinee's eye. Notably, in the present embodiment, the threshold Θ is set to about ⅛ of the size of the image Ln (that is, (Θx, Θy)≈(w/8, h/8)). However, the threshold Θ can suitably be set in accordance with a relationship with desired accuracy. Notably, in the present embodiment, the dataset is divided at an occasion when one of an x component and a y component of the integrated value S exceeded a threshold Θx or Θy. Notably, in the present embodiment, the positional displacement amount dn is the displacement in the image-taken range between two images that are taken sequentially, however, no limitation is made hereto. For example, a displacement of the image-taken range between the selected image and a lead image of the dataset F in which the selected image is included may be used. In this case, the dataset can be divided at an occasion when the positional displacement amount dn exceeds the threshold Θ.

By proceeding to FIG. 8, the description of the flowchart will be continued. Next, the CPU 2 selects a dataset Fs including the most images from among the datasets F1, F2, . . . , Fq created by the process of S29 (S30). Thus, in the process of S30, a plurality of images taken when the fixation of the examinee's eye is most stabilized is selected.

Next, the CPU 2 acquires a gravity center position C of the dataset Fs selected in the process of S30 (S31). The gravity center position C can be obtained from gravity center positions cn of the fundus image portion in the respective images Gn included in the dataset Fs. For example, the gravity center position C can be obtained by dividing an integrated value of the gravity centers cn of the respective images by a number of the images.

Incidentally, each of the images Gn included in the dataset Fs in the present embodiment has a size with a lateral width Mw and a vertical width Mh. In the process of S32, the CPU 2 trims each of the images Gn included in the dataset Fs with the size of a lateral width w and a vertical width h with the gravity center position C as the center (S32). As a result, a dataset Oa=[Oa1, Oa2, . . . , Oap] configured of images On with the lateral width w and the vertical width h is created.

Next, the CPU 2 creates a base image Ob by averaging the respective images included in the dataset Oa (S33). In the base image Ob, a distortion by the involuntary eye movement during fixation included in the respective images of the dataset Oa is averaged. The base image Ob is used as a template of the image processing in a subsequent calibration process (S34).

Next, the CPU 2 performs the calibration process (S34). In the calibration process, the CPU 2 corrects the distortion in the respective images included in the dataset Oa by using the base image Ob as the reference (template). Various methods can be used for the distortion correction. For example, a local region of each image included in the dataset Oa is converted to match the base image Ob. Such a correction method is described in documents (for example, A. Dubra, & Z. Harvey, Registration of 2D Images from Fast Scanning Ophthalmic Instruments. Carlos. O. & S. Sorzano et al, Elastic Registration of Biological Images Using Vector-Spline Regularicalization, and the like). Due to this, an image dataset O=[O1, O2, . . . , Op] formed of images that overlay with high accuracy is created. The process proceeds to the analysis data generating process (see FIG. 6) after the execution of the calibration process, and the CPU 2 continues with the process from S13.

Returning to FIG. 6, the description will be continued. The CPU 2 subjects the images included in the image dataset O to the averaging process, and creates a still image (S13)

Next, the CPU 2 performs an optical distortion correcting process (S14). Due to this, an optical image distortion of the examinee's eye and the ophthalmic imaging apparatus 100 and the like are corrected in the still image created in the process of S13.

Next, the CPU 2 performs a reliability acquiring process (S15). In the process of S15, the CPU 2 acquires reliability of the still image of which image distortion has been corrected. The reliability is information indicating reliability (or validity) of an analysis result derived from an analysis using the still image. The reliability may be information that indicates whether it is an image with high reliability or not, and may be information indicating a degree of the reliability (for example, a numerical value and the like). The reliability becomes a yardstick for a user to select an image for observation and comparison. Generally, the reliability is higher with higher image quality of the still image. Thus, for example, the CPU 2 can acquire the reliability from information such as contrast, brightness and the like of the still image. For example, the reliability is higher with larger contrast. Thus, for example, the CPU 2 may acquire the reliability by using a distribution of the contrast in the still image.

Incidentally, a factor by which an image quality of the still image is degraded (factor by which the reliability becomes low) includes those caused by a situation upon taking the image such as the involuntary eye movement during fixation, or a device setting and the like, and those caused by individual differences in the examinee's eye such as a pupil diameter, eye aberration, clouding of an ocular media and the like. If the low reliability is caused by the situation upon taking the image, the image can be taken again. On the other hand, if the low reliability is caused by the individual differences in the examinee's eye, there are cases where the user may want to select the image as the image to be used for observation and comparison despite the image being a still image with low reliability. Thus, for example, the CPU 2 may acquire reliability that considers the individual differences in the examinee's eye based on at least one of information indicating the pupil diameter, eye aberration, and clouding of the ocular media and the like in the process of S15. Notably, in the present embodiment, as the information indicating the pupil diameter and a degree of the clouding of the ocular media, values that are measured in advance by an ophthalmology device other than the ophthalmology device 100 can be used. Further, the degree of the clouding of the ocular media can be obtained from a profile of a PSF (Point Spread Function) image at an imaging position. IN this case, for example, the PC 1 may have a PSF image of the same imaging position as the fundus image acquired by the ophthalmic imaging apparatus 100 transferred thereto in advance.

Next, the CPU 2 performs the photoreceptor cell analysis processing (S16). In the photoreceptor cell analysis processing of the present embodiment, the CPU 2 detects a photoreceptor cell from the still image corrected by the optical distortion correcting process (S14). A photoreceptor cell point is set for the photoreceptor cell detected from the still image corrected by the optical distortion correcting process (S14). Due to this, in the present embodiment, an analysis result image is created. Notably, the analysis result image only needs to be an image that can be used in analysis, inspection, or comparison and the like with other images, and the photoreceptor cell point does not necessarily need to be set. Further, by using the analysis result image, a photoreceptor cell density, a hexagonal cell incidence, a regular hexagonal cell incidence, and the like as an entirety of the analysis result image are calculated. The image data of the analysis result image and the calculated various analysis results are stored in the HDD 5 (S17).

After the execution of S17, the CPU 2 determines whether all of the groups of images selected by the user in the data list 31 and the like have been processed or not (S18). If there are unprocessed groups of images left (S18: No), the CPU 2 returns to the process of S11, and repeatedly performs the processes of S11 to S18. On the other hand, if all of the groups of images are processed (S18: Yes), the CPU 2 ends the analysis data generating process.

As described above, in the PC 1 of the present embodiment, the dataset Fs (image set Fs) including a plurality of examinee's eye image taken sequentially when the fixation is stabilized is acquired, since it is composed in the base image (S30). The plurality of examinee's eye image taken sequentially when the fixation is stabilized has less differences between images compared to the examinee's eye images taken when the fixation is unstable. Due to this, a satisfactory base image Ob tends to be generated by composing the plurality of examinee's eye images included in the dataset Fs acquired in the process of S30. For example, a distortion in a direction along a retina of the examinee's eye and a distortion in a direction intersecting the retina based on the involuntary eye movement during fixation are more likely to be suppressed in the base image Ob. Thus, the image processing on the examinee's eye image that uses the base image Ob generate in the PC 1 as the template (for example, distortion correction, positioning and the like on the examinee's eye image) is more likely carried out properly. Thus, according to the PC 1, a base image Ob suitable for the template of the image processing can be obtained.

Further, in the PC 1 of the present embodiment, a region (an area to be cut out from each of the examinee's eye images) to be composed to the base image relative to the dataset Fs including the plurality of examinee's eye images that were positioned relative to one another by the process of S23 is set by the CPU 2 (S32). The region to be composed to the base image in the respective examinee's eye images in the dataset Fs had its positional displacement corrected, so the PC 1 is likely to generate a satisfactory base image Ob.

Further, the region to be composed to the base image is set around the gravity center position of the dataset Fs in the state of having been positioned by the process of S23 by the CPU 2 (S32). Due to this, in the respective examinee's eye images included in the dataset Fs, the regions to be composed with the base image tends to be wider. Thus, even more satisfactory base image may be generated. Notably, the regions to be composed to the base image are not limited to regions set with the gravity center position of the dataset Fs as the center, as in the present embodiment.

Further, in the process of S31, the dataset with the largest number of the examinee's eye images taken sequentially is at least acquired from among the plurality of datasets F1, F2, . . . , Fq. The dataset with which fixation of the examinee's eye had been stabilized upon taking the images has larger number of examinee's eye images included in the image set. Due to this, a satisfactory base image tends to be generated from the dataset having the largest number of the examinee's eye images taken sequentially.

Incidentally, supposedly in respectively positioning the examinee's eye images to the reference image Rn (second base image) in the process of S23, if the displacement in the taken positions between the examinee's eye image Ln and the reference image Rn is large, there is a risk that the positioning of the examinee's eye image Ln and the reference image Rn is not appropriately performed. For example, if there are few regions overlapping one another between the examinee's eye image Ln and the reference image Rn, the reliability of the positioning becomes low. Due to this, there is a risk that a satisfactory base image may not be generated.

With respect to this, in the present embodiment, the reference image Rn is updated using the positioned examinee's eye image Ln each time one examinee's eye image is positioned. Thus, the reference image Rn includes information on the plurality of examinee's eye images Ln with different taken positions. Due to this, the overlapping regions between the examinee's eye image Ln and the reference image Rn are more easily secured. Accordingly, the positioning of the examinee's eye images Ln relative to the reference image Rn is likely to be performed satisfactorily.

Notably, the reference image Rn may be updated using at least one of a predetermined number of examinee's eye images Ln each time the predetermined number of examinee's eye images are positioned. In this case, compared to the present embodiment, the frequency of the update of the reference image Rn can be made less. Thus, such a decrease enables the base image to be generated in a shorter period of time.

Further, in the present embodiment, in the case where the examinee's eye image Ln is positioned relative to the reference image Rn (S23), the reference image Rn is in the state in which the examinee's eye image Ln−1 taken sequentially with the examinee's eye image Ln is included just before the update (S27). Due to this, the examinee's eye images Ln−1, Ln that were sequentially taken are unlikely to be affected by an influence of the displacement in the taken positions caused by the involuntary eye movement during fixation. Due to this, the region overlapping between the examinee's eye image Ln and the reference image Rn is more easily secured. Thus, in the PC 1, the positioning of the examinee's eye image Ln relative to the reference image Rn is more likely to be performed in further satisfaction.

<ROI Settings>

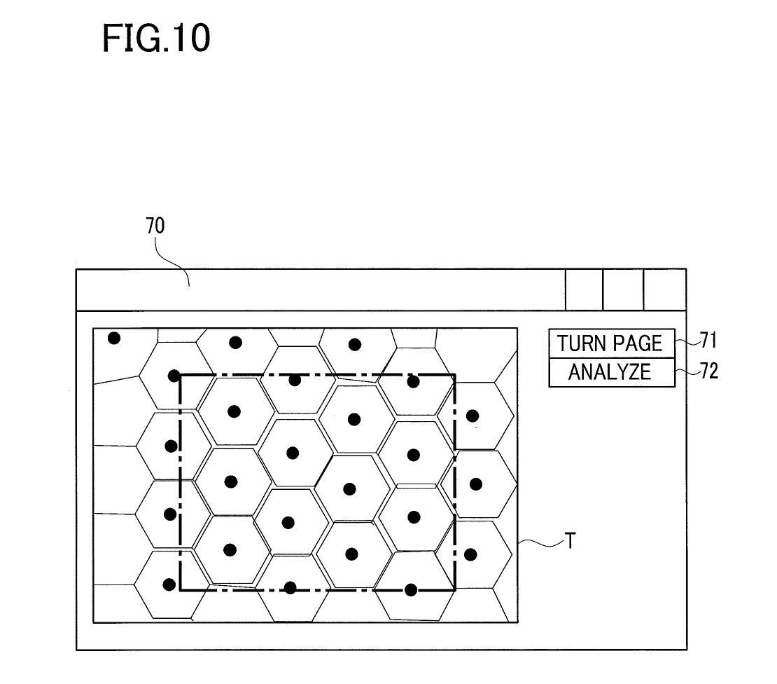

The PC 1 of the present embodiment has prepared therein functions to correct and re-analyze the analysis data by using the analysis result image created in the aforementioned analysis data generating process (see FIG. 6). For example, a target of the analysis in the analysis result image can be changed in accordance with a user's instruction, and analysis can be performed thereon again. For example, the user can designate the area to be used in the analysis within the analysis result image (that is, ROI: Region of Interest) and perform re-analysis. In a case where one or more analysis result images are selected in the data list 31 and the like and a “SET ROI” button 32d in the control box 32 is operated, the CPU 2 displays an ROI setting window 70 as shown in FIG. 10 on the controller 20.

As shown in FIG. 10, in the ROI setting window 70, the user can designate ROI on the analysis result image displayed in an image display region T. The CPU 2 sets the ROI within the range designated by the user. In the present embodiment, the range in which the ROI is set is shown by a one-dot chain line.

In a case where a plurality of analysis result images is selected in advance in the data list 31 and the like, the CPU 2 displays another one of the selected analysis result images in the image display region T based on an operation of a “TURN PAGE” button 71. Further, in a case where an “ANALYZE” button 72 was operated, the CPU 2 performs a re-analysis of the analysis result image selected in the data list 31 and the like. In the present embodiment, in the re-analysis, same process as the photoreceptor cell analysis processing included in the analysis data generating process (see FIG. 6) is performed. In a case where the ROI is set in the analysis result image, a fundus tissue included in the ROI becomes the analysis target. Thus, according to the PC 1, an appropriate analysis result is more likely to be obtained by the re-analysis by setting the ROI by excluding portions where the photoreceptor cell is difficult to detect (for example, blood vessels). Notably, similar re-analysis is performed even if another “ANALYZE” button prepared in the control box 32 and the like is operated.

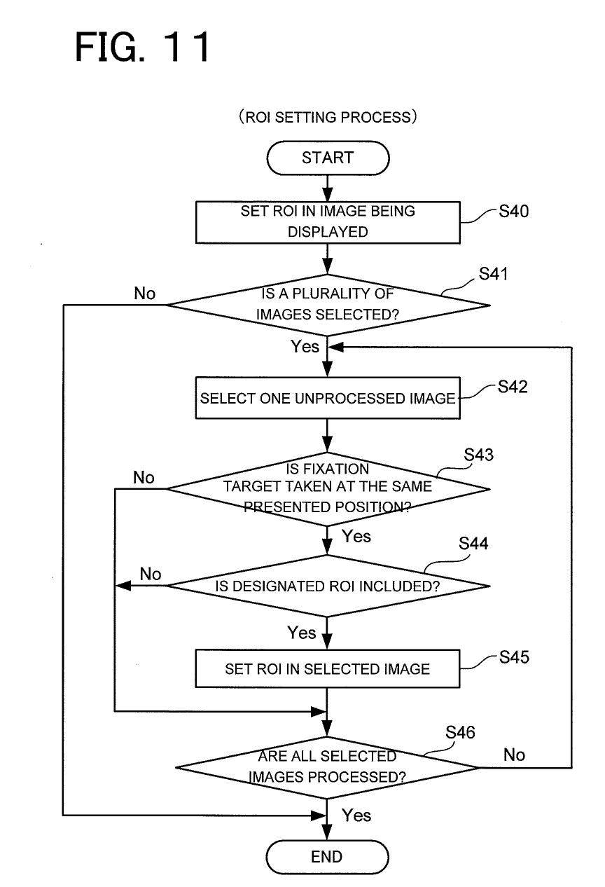

Further, in the present embodiment, in the case where the plurality of analysis result images is selected in advance in the data list 31 and the like, the ROI can collectively be set for the plurality of images. In the case where the ROI is designated by the user for the analysis result image, the CPU 2 performs a ROI setting process (see FIG. 11).

In the ROI setting process shown in FIG. 11, firstly, the CPU 2 sets the ROI in the range designated by the user in the analysis result image being displayed (S40). Next, the CPU 2 determines whether the plurality of analysis result images is selected in advance in the data list 31 and the like or not (S41). If only one analysis result image is selected (S41: No), the CPU 2 skips the processes of S 42 to S46 and ends the ROI setting process. On the other hand, if a plurality of analysis result images is selected in advance (S41: Yes), the process proceeds to the process of S42.

In the process of S42, the CPU 2 selects one image that has not yet been processed through S43 and subsequent steps to be described later (S42). Next, the CPU 2 determines whether the image to which the ROI was set in the process of S40 and the image selected in the process of S42 are taken with the same presented position of the fixation target or not (S43). This determination can be performed for example by comparing information indicating the presented position of the fixation target upon taking the image included in the image data of one another. If the presented position of the fixation target upon taking the image is different between the images (S43: No), the process proceeds to the process of S46 to be described later.

On the other hand, if the presented position of the fixation target upon taking the image is the same (S43: Yes), the process proceeds to the process of S44. In the process of S44, the CPU 2 determines whether the fundus tissue in the ROI set in the process of S40 is included in the image selected in the process of S42 or not (S44). For example, the determination of S44 can be performed based on correlation values that are sequentially calculated while a region in the ROI in the analysis result image being displayed is displaced relative to the image selected in the process of S42 by at least one of the parallel shifting and the rotative shifting. For example, if the maximum value of the correlation value exceeds a predetermined threshold, it is determined that the fundus tissue in the ROI set in the process of S40 is included in the image selected in the process of S42. Further, in the process of S44, the determination of S44 can be performed based on a result of pattern matching of characteristics extracted from both the region in the ROI in the analysis result image that is being displayed and the image selected in the process of S42.

In the process of S44, if the fundus tissue in the ROI set in the analysis result image that is being displayed is not included in the image selected in the process of S42 (S44: No), the process proceeds to the process of S46. On the other hand, if the fundus tissue in the ROI set in the process of S40 is included in the image selected in the process of S42 (S44: Yes), the CPU 2 sets the ROI to the image selected in the process of S42 (S45). In the process of S45, the ROI is set to the same portion as the portion where the ROI was set in the analysis result image that is being displayed.

In the process of S46, a determination on whether all of the images selected in the data list 31 and the like have been processed or not is made by the CPU 2 (S46). If there is an image on which S43 and subsequent processes have not yet been performed in the images selected in the data list 31 and the like (S46: No), the process proceeds to S42 and performs S42 and subsequent processes again. On the other hand, if S43 and subsequent processes have been performed on all of the images selected in the data list 31 and the like (S46: Yes), the process is ended.

<Correction of Photoreceptor Cell Detection Result>

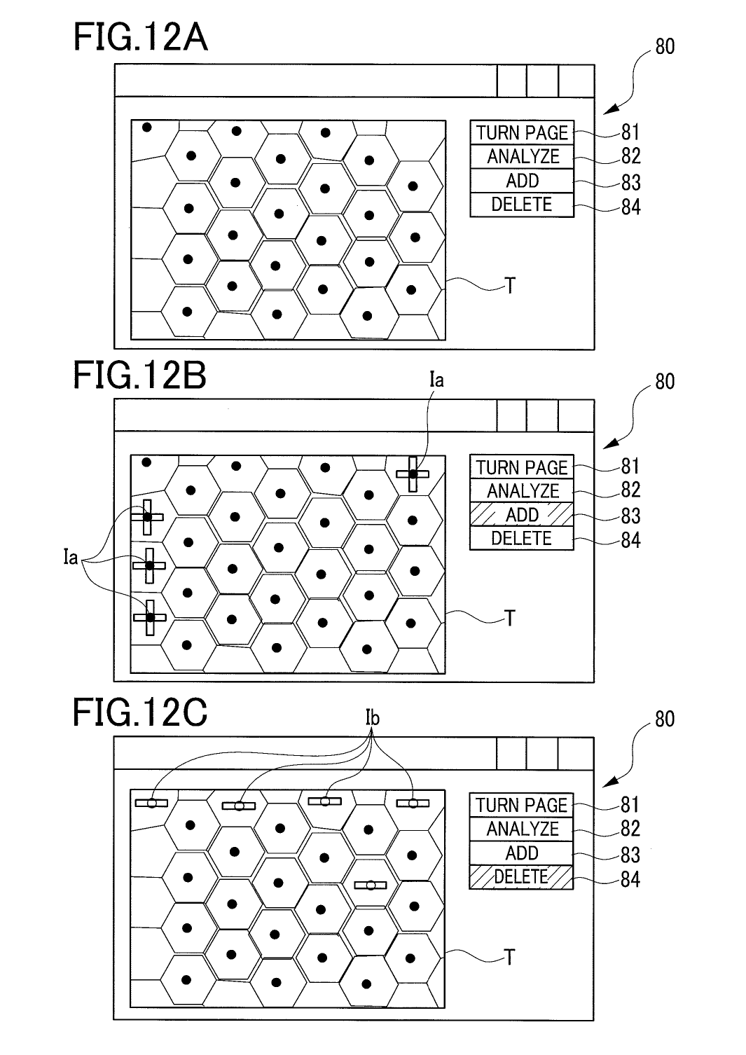

Further, in the PC 1 of the present embodiment, the target to be analyzed in the analysis result image can be changed also by correcting the detection result of the photoreceptor cell in the analysis result image. The correction of the photoreceptor cell detection result is performed on a photoreceptor cell point correcting window 80 shown in FIGS. 12A to 12C. In the case where one or more analysis result images are selected in the data list 31 and the like, and a “PHOTORECEPTOR CELL POINT CORRECTION” button 32e of the control box 32 is operated, the CPU 2 displays the photoreceptor cell point correcting window 80 in the controller 20.

As shown in FIG. 12A, the photoreceptor cell point correcting window 80 has one of the analysis result images selected in the data list 31 and the like is displayed in the image display region T. A role of a “TURN PAGE” button 81 is the same as the other “TURN PAGE” button described earlier.

As shown in FIG. 12B, when an “ADD” button 83 is operated by the user, the user can instruct a position where a photoreceptor cell point is to be added in the analysis result image. When the position where the photoreceptor cell point is to be added is instructed by the user, the CPU 2 sets an icon Ia at the position on the analysis result image instructed by the user. On the other hand, as shown in FIG. 12C, when a “DELETE” button 84 is operated by the user, the user can instruct a photoreceptor cell point to be deleted from the analysis result image. When the photoreceptor cell point to be deleted is instructed by the user, the CPU 2 sets an icon Ib at the position of the photoreceptor cell point deleted by the user.

When an “ANALYZE” button 82 is operated in a state where one of the icon Ia and the icon Ib is set, the CPU 2 corrects the photoreceptor cell point on the analysis result image that is being displayed. For example, the CPU 2 adds a photoreceptor cell point to a position where the icon Ia is seta On the other hand, the CPU 2 deletes the photoreceptor cell point at a position where the icon Ib is set. An image in which the photoreceptor cell point has been corrected is stored in the HDD 5 as a new analysis result image. Further, the CPU 2 performs processes similar to the photoreceptor cell analysis processing included in the analysis data generating process (see FIG. 6) on the new analysis result image. Due to this, the respective analysis results on the new analysis result image are outputted.

Notably, similar to the case of selecting the plurality of analysis result images and setting the ROI, in the case of selecting the plurality of analysis result images and correcting the detection results of the photoreceptor cell points also, the CPU 2 can reflect the correction of the photoreceptor cell point performed on one analysis result image to other analysis result images having the same photoreceptor cell point.

<Change in Information on Eye Axis Length>

In the PC 1 of the present embodiment, the re-analysis of the examinee's eye can be performed by changing information on eye axis length of the examinee's eye. The user inputs an eye axis length of the examinee's eye in an eye axis length input box 32g (see FIG. 3) of the control box 32, and selects one or more analysis result images in the data list 31 and the like, and then operates an “ANALYZE” button 32c. Due to this, the CPU 2 performs re-analysis of the selected analysis result image based on the eye axis length inputted in the eye axis length input box 32g. Here, a process similar to the photoreceptor cell analysis processing included in the analysis data generating process (see FIG. 6) is performed under the changed eye axis length. With the information on the eye axis length of the examinee's eye being changed, an estimated value of a size of the photoreceptor cell is changed in the photoreceptor cell analysis processing. Thus, the analysis result on a more accurate photoreceptor cell density can be obtained by inputting the accurate eye axis length measured by an eye axis length measuring device and the like in the eye axis length input box 32g.

In the present embodiment, a case in which the eye axis length of the examinee's eye is changed in performing the re-analysis was described, however, other methods may be employed so long as the accurate size of the photoreceptor cell can be used in the re-analysis. For example, a curvature radius of a cornea of the examinee's eye may be configured changeable in performing the re-analysis. For example, the user can input the curvature radius of the cornea similar to inputting the eye axis length in the eye axis length input box 32g in the present embodiment. Notably, only one of the eye axis length and the curvature radius of the cornea may be configured changeable, however, by configuring to change both of them, more accurate size of the photoreceptor cell can be used in the re-analysis. Thus, in this case, more appropriate analysis result can be obtained.

<Follow-Up Display>

The PC 1 of the present embodiment has a follow-up function that can display images with different image-taking dates in the same arrangement on the same screen (that is, concurrently). By the follow-up display, the user can compare how the specified position of the fundus has changed over time.

A setting of a baseline image is enabled by the user operating a “SET BASE IMAGE” button 32f in the control box 32 (see FIG. 3). The baseline image is an image to be used as a reference in the comparison. In the present embodiment, firstly, after the user operated the “SET BASE IMAGE” button 32f, the user is caused to select a file name of one of the analysis result images aligned in the data list 31. Due to this, the image with the file name selected by the user is set as the baseline image by the CPU 2. Next, the user is caused to check one or more of the check boxes 31a of the analysis result images other than the baseline image. Due to this, the images checked by the user are set as comparison images to be concurrently displayed with the baseline image by the CPU 2. Notably, the baseline image and the comparison images may be selected from the wide-field list window 60 shown in FIGS. 5A, 5B. The user can easily select the baseline image and the comparison images since thumbnail images of the analysis result images taken at the same image-taking position are displayed at the same position in the wide field fundus image W.

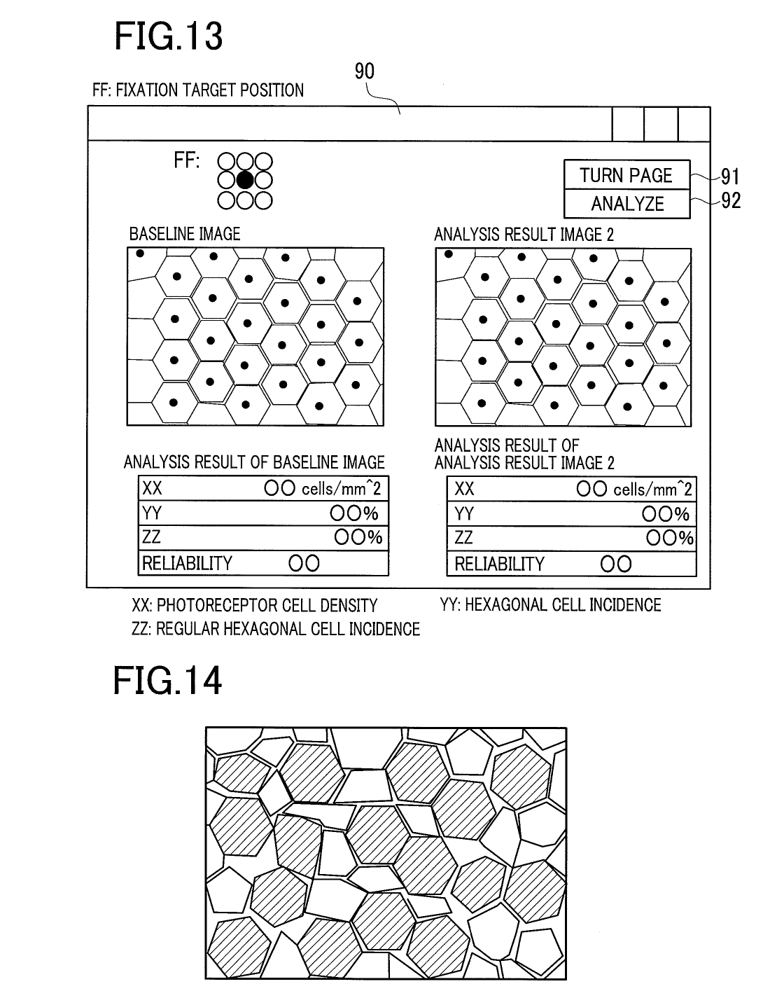

In a state in which the baseline image and the comparison images are set, when the “ANALYZE” button 32c is operated by the user, the CPU 2 displays a follow-up display window 90 (see FIG. 13). As shown in FIG. 13, the follow-up display window 90 has the baseline image and at least one of the comparison images displayed therein. Notably, in FIG. 13, the analysis result image 2 is displayed as the comparison image. The comparison image is displayed in the follow-up display window 90 in a state of having been positioned relative to the baseline image. The positioning is performed by the CPU 2 moving the comparison image by rotative shifting and parallel shifting relative to the baseline image. For example, in a case where a correlation of the baseline image and the comparison image is calculated, the comparison image simply needs to be moved to a position with the highest correlation value.

Further, in the follow-up display window 90, analysis results of the baseline image and the comparison images are respectively displayed by the CPU 2. For example, the analysis results of the respective images that are stored in the HDD 5 in advance may be displayed. However, if the baseline image and the comparison image have displaced image-taking positions, it becomes difficult to accurately compare the analysis results of the photoreceptor cell density and the like of the baseline image and the comparison image. Thus, in the present embodiment, a common ROI may be set in the baseline image and the comparison image and perform the re-analysis. For example, similar to the aforementioned ROI setting window 70 (see FIG. 10), the user may be enabled to designate the ROI on the baseline image (or the comparison image) in the follow-up display window 90. For example, in a case where the “ANALYZE” button 92 is operated after the ROI is designated in the baseline image, the CPU 2 performs the aforementioned ROI setting process on both the baseline image and the comparison image. Due to this, the ROI can be set in the common region in the baseline image and the comparison image. A region where a common fundus tissue is taken may be searched, and the ROI may be set in the region that is common in the respective images. When the common ROI is set in both the baseline image and the comparison image, the CPU 2 calculates the analysis results of the cell density and the like, and outputs the analysis results on a screen and the like. Due to this, it becomes easier for the user to compare the analysis results on the photoreceptor cell density and the like between the baseline image and the comparison image.

Further, as shown in FIG. 13, the follow-up display window 90 has reliability of each image displayed therein. The user can select the image to be compared in accordance with the reliability. Thus, the user can appropriately compare the images.

Further, as shown in FIG. 14, in the baseline image and the comparison images, hexagonal cells may be displayed in a manner different from cells with other shapes. For example, the hexagonal cells may be colored differently from the cells with other shapes, or hatching may be applied thereto. A healthy retina has hexagonal cells arranged regularly. The hexagonal cells are known to increase its corners in the course of its shape being deformed due to abnormalities such as pathological change and the like. Alternatively, among the hexagonal cells, regular hexagonal cells may be displayed in a manner different from cells other than the regular hexagonal cells.

As described above, in the PC 1 of the present embodiment, in the case where the ROI setting window 70, the follow-up window 90, and the like are being displayed, the instruction from the user to change the target to be analyzed by the analysis processing of the photoreceptor cell in the plurality of fundus images is received by the CPU 2 via the operation unit 14 and the operation processing unit 8. When the analysis processing of the photoreceptor cell is performed in a case where the target related to the instruction received by the CPU 2 is included in the overlapping portion of the plurality of images (S44: Yes), the analysis results that reflected the instruction of the user for each of the plurality of images are outputted by the CPU 2. Accordingly, the target to be analyzed in the plurality of images is collectively changed by the instruction from the user. Thus, burden on the user who instructs to change the analysis target in the case of analyzing the plurality of images having the overlapping portion at least in parts of each other is likely to be suppressed. Especially, in the present embodiment, the CPU 2 receives the instruction from the user by using one of the images displayed in the ROI setting window 70, the follow-up window 90, and the like. Due to this, the user can easily instruct to change the analysis target.

Further, in the present embodiment, in a case where the ROI instructed by the user is received by the CPU 2, a fundus tissue within the ROI set in each of the plurality of images is analyzed by the CPU 2. Thus, the analysis at a range that the user desires can be performed in each of the plurality of images while suppressing the burden on the user.

As above, the description was given based on the embodiment, but the present disclosure is not limited to the above embodiment, and can be modified in various ways.

For example, in the above embodiment, the movement of the examinee's eye upon taking the images was detected by causing the CPU 2 to calculate the displacement amounts of the taken positions between serially taken images. However, the movement of the examinee's eye upon taking the images may be detected by other ways. For example, wide field fundus images (front views of the fundus) are sequentially taken by the second imaging unit 105 during when one group of images is being taken by the fundus imaging optical system 101 of the ophthalmic imaging apparatus 100. The PC 1 is caused to acquire the wide field fundus images that were sequentially taken together with the group of images. Thereupon, the CPU 2 may detect the movement of the examinee's eye upon taking the group of images based on a movement of a specific portion such as blood vessels, macula, and the like shown in the wide field fundus images that were sequentially taken. Further, when the examinee's eye moves, the aberration detected by the wave front sensor 102 changes. Thus, for example, the aberration (mainly the wave front aberration by the examinee's eye) detected by the wave front sensor 102 during when the one group of images is taken by the fundus imaging optical system 101 is acquired successively by the ophthalmic imaging apparatus 100. The PC 1 is caused to acquire the detection results of the aberration during when the group of images is taken together with the group of images. Thereupon, in the PC 1, the movement of the examinee's eye upon taking the group of images may be detected by the CPU 2 based on the detection results of the aberration. By using either methods, the movement of the examinee's eye upon taking the group of images can be detected without needing any special device.

Further, in the analysis data generating process of the above embodiment, one set of analysis data (analysis result image and analysis data of the photoreceptor cell density, and the like) is obtained from one group of images. However, one set of analysis data may be generated from a plurality of groups of images. For example, in a case where a plurality of groups of images taken at the same position of the examinee's eye on the same image-taking day is selected in the data list 31 and the like, the selected plurality of groups of images may be regarded as one group of images, and the analysis data generating process (See FIG. 6) may be performed thereon. Assumingly, if a plurality of analysis data of the same image-taking day exists, if would be difficult for the user to determined which data should be used. With respect to this, in a case where the analysis data generating process is performed by regarding the plurality of groups taken at the same position of the examinee's eye on the same image-taking day as one group of images, the analysis data for the occasion during that day when the fixation was most stabilized can be obtained.

Further, in the above embodiment, the case in which the base images for each group of images is independently generated in the case where the plurality of groups of images existed was described. However, no limitation is made necessarily hereto, and the base image generated by using an image included in one group of images may not only be used as the template of the image processing on this one group of images, but also may be used as the template of the image processing for a base image of another group of images having the same image-taken position as the one group of images. For example, a base image Ob1 generated from a first group of images may be used as the template in the case of performing positioning and the like of a second group of images having a different image-taking date from the first group of images. In this case, for example, a dataset f2 with a stabilized fixation in the second group of images is positioned relative to the base image Ob1 by the CPU 2, and distortion thereof is corrected. Moreover, the CPU 2 cuts out the dataset f2 in the range of the base image Ob1 to generate an analysis result image. Due to this, the analysis result image generated from the first group of images and the analysis result image generated from the second group of images use the same base image as their templates, so the user can easily compare the analysis results. Further, in the case of collectively changing the analysis target (for example, the ROI and the like) of a plurality of analysis result images, the analysis target after the change is more preferably set to each image by the respective analysis result images being generated using the same base image as their templates. As a result, the operational burden on the user can preferably be reduced.

In the above embodiment, the case in which the reliability of the images is calculated for the images that were subjected to averaging in the process of S13, however, no limitation is made hereto. For example, the reliability of the images before adding the images may be calculated.

Further, in the above embodiment, in the case where the ROI is set in a plurality of analysis result images by the ROI setting process (see FIG. 11), the region where the ROI is set by the user for one analysis result image was searched by the CPU 2 in other analysis result images as well.

The CPU 2 then had set the ROI in the searched regions in the other analysis result images. However, the region where the ROI is set by the user for one analysis result image may not necessarily have to be searched by the CPU 2 in the other analysis result images. For example, in a case where a difference in the image-taken range between the analysis result images is sufficiently small, the CPU 2 may set the ROI of the other analysis result images at the same position (coordinates) on the images as the ROI instructed by the user.

Notably, in the above embodiment, in the PC 1, the case of processing the fundus images that are taken by the AO-SLO as the ophthalmic imaging apparatus 100 was described. However, according to the present disclosure, images taken by various types of devices other than the AO-SLO that can take pictures of the examinee's eye can be processed in the PC 1. For example, an Optical Coherence Tomography (OCT) that acquires tomographic images at an anterior segment or fundus may be used as the ophthalmic imaging apparatus 100.

Although the present disclosure was described with reference to a specific embodiment by referring to the drawings, the present disclosure is not limited thereto, and it should be understood that the present disclosure encompasses all of possible alterations and modifications that can be made without going beyond the essence of the present disclosure as defined by the claims attached herewith.

### REFERENCE SINGS LIST

1 PC
2 CPU
5 HDD
13 Monitor
15 External memory
Ob Base image
W Wide field fundus image

What is claimed is:

1. An image processing apparatus including:
an analyzer configured to process a plurality of examinee's eye images taken from a same examinee's eye, at least a part of the examinee's eye images being overlapped with each other, and output an analysis result of a cell of the examinee's eye for each of the examinee's eye images; and
an instruction receiving unit configured to receive an instruction regarding a target to be analyzed by the analyzer in the plurality of examinee's eye images from an examiner,
wherein the analyzer outputs the analysis results in which the instruction received by the instruction receiving unit is reflected for each of the plurality of examinee's eye images.

2. The image processing apparatus according to claim 1, further including a display control unit configured to display one of the plurality of examinee's eye images on a display device,
wherein the instruction receiving unit receives the instruction from the examiner via one examinee's eye image displayed by the display control unit, and
in a case where the instruction is received by the instruction receiving unit, the analyzer outputs the analysis results in which the instruction is reflected for each of the plurality of examinee's eye images.

3. The image processing apparatus according to claim 1,
wherein the instruction receiving unit receives an instruction from the examiner instructing a region to be analyzed by the analyzer in the examinee's eye, and
the analyzer analyzes the region instructed by the instruction received by the instruction receiving unit for each of the plurality of examinee's eye images.

4. The image processing apparatus according to claim 1,
wherein the analyzer detects the cell of the examinee's eye included in the examinee's eye images and analyzes the examinee's eye images by using the detection results,
the instruction receiving unit receives an instruction to correct the detection results, and
in a case where the instruction is received by the instruction receiving unit, the analyzer further corrects the detection result of each of the plurality of examinee's eye images in accordance with the instruction.

5. The image processing apparatus according to claim 2, wherein the display control unit displays the analysis results outputted from the analyzer for at least two or more examinee's eye images taken on different days on a same screen of the display device.

6. The image processing apparatus according to claim 2,
wherein the plurality of examinee's eye images are taken at a same location of the examinee's eye,
the display control unit is configured to:
display a list of one or more image indexes, each of which indicating a group of images including the plurality of examinee's eye images, on a display screen of the display device based on information indicating taken positions of the groups of images, and display, on the display screen, a wide field examinee's eye image taken by a wider range than each of the examinee's eye images, and
arrange each image index indicating a group of images at the taken position of the group of images in the wide field examinee's eye image in a manner of being overlaid on the wide field examinee's eye image.

7. The image processing apparatus according to claim 6,
wherein the instruction receiving unit selects at least one of the plurality of image indexes displayed on the display screen by the display control unit in accordance with an instruction from the examiner, and
the analyzer performs image processing on the group of images indicated by the image index selected by the instruction receiving unit.

8. The image processing apparatus according to claim 7,
wherein in a case of performing the image processing on the groups of images indicated by the image indexes, the analyzer generates, in association with the image index for which the image processing had been performed, processed information indicating that the image processing had already been performed, and
the display control unit adds, on each of the image indexes, a display indicating whether the image processing had already been performed on the group of images indicated by each of the image indexes displayed on the display screen or not in accordance with the processed information.

9. The image processing apparatus according to claim 7,
wherein the instruction receiving unit selects which of a first image index, with which the image processing on the group of images had been performed, or a second image index, with which the image processing has not yet been performed, is to be displayed on the display screen based on an operation from the examiner, and
the display control unit displays, on the display screen, one of the first image index and the second image index that was selected by the instruction receiving unit.

10. The image processing apparatus according to claim 6,
wherein a group of images indicated by the image index includes a plurality of images of the examinee's eye taken in a state of being fixed by using a fixation target, and
the display control unit switches the image index to be displayed on the display screen for each of a fixation target position upon taking the group of images indicated by each of the image index.

11. The image processing apparatus according to claim 6, wherein the display control unit displays, on the display screen, the wide field examinee's eye image taken at a same fixation position as the group of images indicated by the image index displayed in the display screen.

12. The image processing apparatus according to claim 1,
wherein an examinee's eye image of which analysis result is obtained by the analyzer is a first examinee's eye image,
the image processing apparatus further includes a motion detecting unit that detects motion of the examinee's eye upon image-taking in a plurality of second examinee's eye images, which is second examinee's eye images stored in a storage device, and in which the same examinee's eye is taken, and
the analyzer is configured to:
acquire an image set including a plurality of second examinee's eye images taken sequentially when a fixation is stabilized from among the plurality of second examinee's eye images stored in the storage device based on a detection result of the motion detecting unit, and
generate a base image, which is to be used as a template in image processing for generating the first examinee's eye image, by composing the plurality of second examinee's eye images included in the image set.

13. The image processing apparatus according to claim 12, wherein the analyzer corrects distortions of the plurality of second examinee's eye images stored in the storage device with the base image as the template, and generates an averaging image including the second examinee's eye images of which distortions have been corrected.

14. The image processing apparatus according to claim 12,
wherein the analyzer is configured to:
perform positioning by overlaying the second examinee's eye images included in the image set relative to each other by at least one of horizontal shifting and rotative shifting, and
set a region, where the second examinee's eye images are composed to each other upon generating the base image, to the image set having each of the second examinee's eye images positioned.

15. The image processing apparatus according to claim 13, wherein the analyzer sets the region around a gravity center position of the image set having each of the second examinee's eye images positioned.

16. The image processing apparatus according to claim 12,
wherein the analyzer is configured to:
at least acquire an image set having a largest number of examinee's eye images that are chronologically sequential, in a case where there are plural sets of the image sets satisfying an acquisition reference based on a detection result of the motion detecting unit.

17. A storage medium storing a computer-readable image processing program,
wherein the image processing program, when executed by a processor of a computer, causes the computer to perform:
an analyzing step of analyzing plurality of examinee's eye images stored in a storage device, the examinee's eye images having taken a same examinee's eye, and outputting an analysis result of a cell of the examinee's eye for each of the images; and
a receiving step of receiving an instruction for changing an analysis condition in the analyzing step from an examiner,
in a case where the instruction is received in the receiving step, the analysis results, in which the analysis condition according to the instruction is reflected, are outputted for each of the plurality of examinee's eye images in the analyzing step.

18. The storage medium according to claim 17,
wherein the plurality of examinee's eye images is a plurality of examiner's eye images taken at a same position of the examinee's eye,
the image processing program further causes the computer to perform:
a list displaying step of displaying a list of a plurality of image indexes, each of which indicating a group of images including the plurality of examinee's eye images, on a display screen of the display device based on information indicating taken positions of the groups of images relative to the examinee's eye;
an index selecting step of selecting at least one of the image indexes displayed on the display screen by the list displaying step, based on an instruction from the examiner;
an image processing step of performing image processing on the group of images indicated by the selected image index; and
a wide field image displaying step of displaying, on the display screen, a wide field examinee's eye image taken by a wider range than each of the images,
in the list displaying step, the image index indicating each group of images is arranged, in a manner of being overlaid on the wide field examinee's eye image, at the taken position of the group of images in the examinee's eye image displayed in the wide field image displaying step.

19. The storage medium according to claim 17,
wherein an examinee's eye image of which analysis result is obtained by the analyzing step is a first examinee's eye image,
the image processing program further causes the computer to perform:
a motion detecting step of detecting motion of the examinee's eye upon taking a plurality of second examinee's eye images stored in a storage device;
an acquiring step of acquiring an image set including a plurality of second examinee's eye images taken sequentially when a fixation is stabilized from among the plurality of second examinee's eye images stored in the storage device based on a detection result of the motion detecting step; and
a base image generating step of generating a base image, which is to be used as a template in image processing for generating the first examinee's eye image, by composing the plurality of second examinee's eye images included in the image set acquired in the acquiring step.
